(19) United States
(12) Patent Application Publication
(10) Pub. No.: US 2011/0301620 A1
(43) Pub. Date: Dec. 8, 2011

(54) DEVICE FOR SUTURING TWO HOLLOW BIOLOGICAL TISSUES

(75) Inventors: Marie Di Betta, Le Plessis Robinson (FR); Christine Rotinat-Libersa, Le Plessis Robinson (FR); Anne-Laure Vayssade, Paris (FR)

(73) Assignee: Commissariat A L'Energie Atomique Et Aux Energies Alternatives, Paris (FR)

(21) Appl. No.: 13/201,599

(22) PCT Filed: Feb. 18, 2010
(86) PCT No.: PCT/EP2010/052021

§ 371 Date: Aug. 15, 2011

(30) Foreign Application Priority Data
Feb. 20, 2009 (FR) 09 51116

(51) Int. Cl. A61B 17/04

(52) U.S. Cl. 606/144

(57) ABSTRACT

The device comprises a first part able to be provided on the free side of one of two hollow biological tissues bearing against each other and a second part able to be provided on the free side of the other of the two hollow biological tissues bearing against each other, the first part comprising two rows of needles enabling the walls of the two hollow biological tissues to be pierced to bring a first suturing thread, and optionally a cut for incising both walls, the second part comprising means for providing a second suturing thread in the loops of the first suturing thread.

### TECHNICAL FIELD

The present invention relates to a device for suturing two hollow biological tissues. It enables, for example, side-to-side anastomoses to be made on a beating heart in mini-invasive surgery to suture a graft on an artery. The invention can also be applied to making an anastomosis between two vessels in side contact with each other or more generally to suturing two hollow biological tissues bearing against each other.

### STATE OF PRIOR ART

A side-to-side vascular anastomosis is an anastomosis placing side by side the side walls of each of both vessels. For example, a graft (a vein taken in a place of the body different from the place where the anastomosis must be provided) can be associated with an artery (for example the coronary artery) so as to bypass the blood flow in order to restore the blood stream obstructed or hampered by stenoses.

U.S. Pat. No. 7,300,444 discloses a device only enabling end-to-side anastomoses to be made by placing staples to fasten a graft on a target vessel. This device enables to set the distance between two staples as well as the number of staples to be implanted depending on the graft end circumference. After the staples are implanted, the apparatus incises the walls of the target vessel using a cutting blade. The length of the incision as well is adjusted depending on the anastomosis size. With this apparatus, the artery is perforated from outside to inside. This perforation direction can be a problem when atheromatous plaques are found on the inner wall of the artery.

The Da Vinci robot from Intuitive Surgical Inc. company can enable anastomoses to be made in mini-invasive surgery, but it makes the suture stitch by stitch, which makes the task slow (it reproduces movements of the surgeon in real time) and does not enable to have mini-invasive access, because of its hindrance, to areas hard to reach as for example those located behind the heart.

Endovascular prostheses or “stents” and graft connectors enable anastomoses to be made by simplifying and speeding up the surgery. Even if most of them are biocompatible, they nevertheless pose reliability and blood clotting problems because they permanently contact blood. Moreover, when placing the stent, the patient must receive an anticoagulant therapy and be regularly monitored. Moreover, placing stents still remains reserved to some categories of patients.

There are suturing aid apparatuses using a thread, but these apparatuses make a suture stitch by stitch because they generally only enable to join flat tissues edge to edge. They are not usable in mini-invasive surgery and are not necessarily designed for making anastomoses.

### SUMMARY OF THE INVENTION

In order to address the problems set out above, the present invention provides a device enabling this operation to be made mini-invasively, including for areas difficult to reach, keeping very close from a result obtained by a manual suturing which would need to open the patient while reducing the operation duration, the recovery time of the patient and the hospitalisation cost.

The device comprises two distinct parts. By way of example, one part may be inserted into an artery (for example the coronary artery) and contains needles, a first thread reserve and a cutter, and another part may be inserted into a graft and comprises two hooks and a second thread reserve.

The device according to the invention has the following advantages:

making a side-to-side anastomosis of a quality equal to or higher than that manually provided by the surgeon,

making an anastomosis on beating heart mini-invasively, while avoiding opening the patient,

enabling to make the incision restoring the blood flow using a cutting means after the suture stitches have been made (the incision is made last),

enabling to use suture stitch,

making several suture stitches in a single manipulation cycle of the apparatus (this is not a suturing stitch by stitch) hence a surgery short duration,

being usable even in areas currently inaccessible or difficult to reach (for example, the back face of the heart).

Thus, the object of the invention is to provide a device for suturing two hollow biological tissues bearing against each other, characterised in that it comprises a first part able to be provided on the free side of one of the two hollow biological tissues and a second part able to be provided on the free side of the other of the two hollow biological tissues, the first part and the second part cooperating together and comprising means for positioning them with respect to each other by grasping walls of the two hollow biological tissues bearing against each other,
the first part comprising:
   a first reserve for a first suturing element,
   needles aligned along two rows located on either side of an axis of the first part, the needles having their sharp ends directed to the second part, the sharp ends of the needles each having means for receiving a loop of the first suturing element, each needle having a hole located so as to lie in the loop of the first suturing element received by the needle and substantially in the loop plane, the hole communicating with an exterior edge of the needle through a slot called outlet channel,
   means for actuating the needles to bring them from a low initial position to a high final position, to pierce the walls of the two biological tissues by the sharp ends of the needles, and then to bring the needles back to their initial position,
the second part comprising:
   a second reserve for a second suturing element,
   driving means for driving a first part of the second suturing element into the holes of the needles in one of the two rows of needles and for driving a second part of the second suturing element into the holes of the needles of the other row of needles when the needles are in the high position.

The first part can also comprise means for cutting the walls of the two biological tissues. These cutting means may be selected from an ultrasound device and a laser device. According to another alternative, they may comprise a cutter the cutting edge of which is directed to a the second part, the cutting means being located between the two rows of needles, the first part comprising means for actuating the cutting means to bring them from a low initial position to a high final position enabling the walls of the two hollow biological tissues to be incised by the sharp edge of the cutting means, and then to bring the cutting means back to the initial position. According to a particular application of the invention, the hollow biological tissues being vessels, the first part of the device is able to be inserted into one of the vessels, the second part of the device is able to be inserted in the other vessel, the device thus enabling a side-to-side anastomosis to be made between both vessels.

The means enabling to position the first part and the second part with respect to each other comprise a female-shaped element formed in one of the parts and a male-shaped element formed in the other part. They also may comprise a magnetic sensor accommodated in the first part and a magnetic sensor accommodated in the second part.

Positioning both parts may also be carried out using a remote imaging device, for example through X-ray photography or ultrasounds, enabling to locate both parts of the device.

The means for actuating the needles can comprise hydraulic actuating means.

The means for actuating the cutter can comprise hydraulic actuating means.

The driving means for driving a first part and a second part of the second suturing element may be actuated by hydraulic actuating means.

According to a particular embodiment, the driving means for driving a first part and a second part of the second suturing element comprise hooks able to draw the second suturing element.

According to another particular embodiment, the driving means for driving a first part and a second part of the second suturing element comprise pushers able to push the second suturing element.

The first suturing element may be selected from a flexible thread an elastic thread and a rigid thread.

The second suturing element may be selected from a flexible thread, an elastic thread and a rigid thread.

Another object of the invention is to provide a needle to be used in a device as described above, comprising a rod the first end of which is able to be fastened in the first part and the second end of which is sharp, the sharp end being provided with a hole, a gutter able to receive the first suturing element and made on the outline of the sharp end and substantially in the hole plane, and a slot communicating the hole with an exterior edge of the needle, the sharp end comprising at least two tips.

The slot may be made in the axis of the rod, whereby the sharp end comprises four tips.

The slot may not be made in the axis of the rod, whereby the sharp end comprises two tips.

At least one of the tips may be longer than the others.

### BRIEF DESCRIPTION OF THE DRAWINGS

The invention will be better understood and other advantages and features will appear upon reading the following description, given by way of non-limiting examples, along with the appended drawings wherein:

FIG. 1 is a view illustrating the operation principle of a device according to the invention,

FIGS. 2A to 2D are also views illustrating the working principle of a device according to the invention,

FIGS. 3, 4 and 5 are different views of a needle usable for the device according to the invention,

FIGS. 6 and 7 are different views of the sharp end of another needle usable for the device according to the invention,

FIGS. 8 and 9 are different views of the sharp end of yet another needle usable for the device according to the invention,

FIG. 10 is an explanatory view representing the thread of the second reserve (or second thread) passing through a loop of the thread of the first reserve (or first thread),

FIG. 11 is another explanatory view showing in cross-section a part of the artery wall placed to the side of a part of the graft wall,

FIG. 12 is a perspective view of a first part of the device according to the invention, showing needles in the high position,

FIG. 13 is a perspective view of a first part of the device according to the invention, showing the cutter in the high position,

FIG. 14 is a perspective view of second part of the device according to the invention, showing the second part during an operating phase,

FIG. 15 is a perspective view of the device according to the invention, showing some elements of the device during an operating phase.

### DETAILED DESCRIPTION OF PARTICULAR EMBODIMENTS

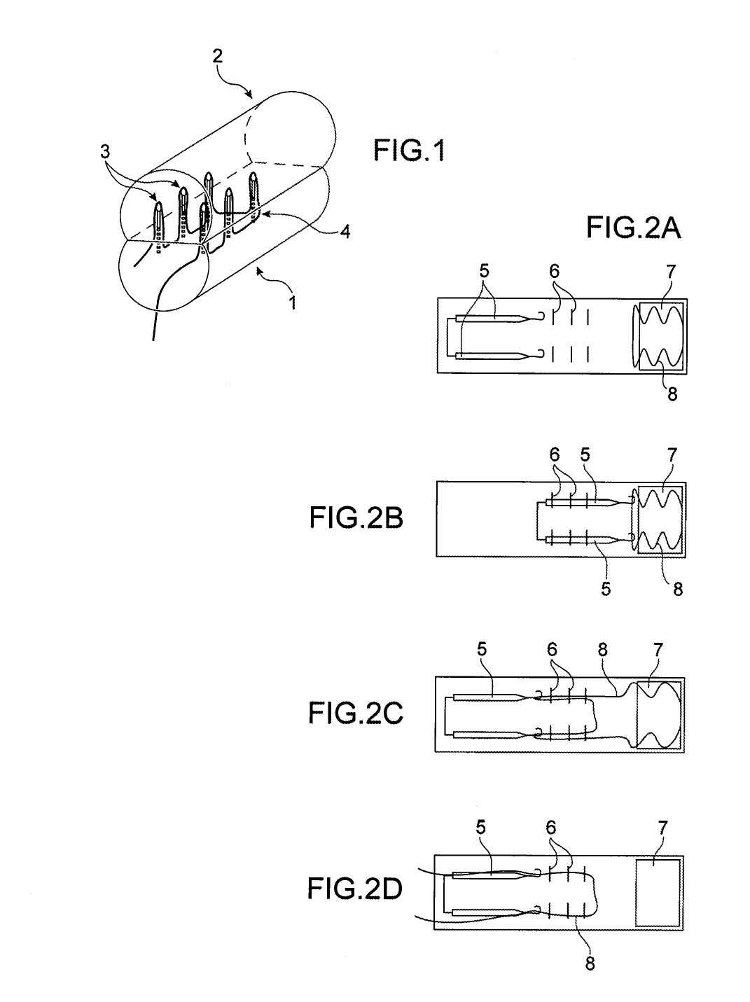

First, for the sake of understanding, the operation principle of the device according to the invention will be described by means of the drawings of FIGS. 1 and 2A to 2D. This principle is herein applied to a particular embodiment of the invention.

FIG. 1 represents an artery 1 and a graft 2 provided in a side-to-side position. The first part of the device is introduced into the artery 1 and the second part of the device is introduced into the graft 2. Both parts of the device are positioned face to face by means of a male shape and a female shape given to the faces facing both parts. When they are engaged with each other, both parts grasp the corresponding walls of the artery 1 and the graft 2 which are located therebetween. These walls are sufficiently thin and flexible to comply with the configuration imposed by the male and female shapes of both parts of the device.

In FIG. 1, the second part of the device (part lying in the graft 2) is not represented. Regarding the first part (which lies in the artery 1), only the needles 3 and a suturing thread 4 are represented. The needles 3 are represented in a high position, that is they have been actuated so that their sharp ends pierce the artery and graft walls grasped by both parts of the device.

The needles 3 are aligned along two rows of three needles and each form, upon rising up, a loop of the suturing thread 4, this thread being initially contained in the reserve of the first part of the device. As clearly shown in FIG. 1, the thread 4 forms a loop towards the tip of each needle and goes from row to row. Both ends of the suturing thread 4 then lie on the same side.

Once the loops are formed in the graft, two hooks belonging to the second part of the device are actuated to pass through the loops of each row and to go and pick up some thread accommodated in a second reserve belonging to the second part and bring back this thread through the loops. This is illustrated by FIGS. 2A to 2D which are top views of the second part.

FIG. 2A shows two hooks 5 which are actuated simultaneously. Each hook 5 is aligned with a row of needles which are not represented in FIGS. 2A to 2D. The hooks 5 are positioned so as to be able to pass through the corresponding loops 6 of the suturing thread (having the reference number 4 in FIG. 1). FIG. 2A shows the second reserve 7 containing a second thread 8. The second thread is provided so as to be gripped by the hooks via its centre part.

FIG. 2B shows the hooks 5 gripping the thread 8 after that each of them has passed through the corresponding loops of its row of needles.

Once the thread 8 is gripped by the hooks 5, the latter returns to their start position by passing again through the loops 6 and by driving the thread 8 with them as shown in FIG. 2C.

The centre part of the thread 8 is returned by the two loops 6 the closest to the second reserve 7 whereas the ends of the thread 8 are brought opposite the second reserve 7. This is shown in FIG. 2D.

The needles are then withdrawn from the graft by bringing them back into the first part of the device. The surgeon can then recover the ends of the threads to tension the suture and make knots by a means known to those skilled in the art. He/she may maintain the threads in this position by another means known to those skilled in the art.

A special needle has been designed to provide the different functions assigned thereto, that is:
piercing the artery and graft walls,
bringing therewith the thread of the first reserve upon piercing the artery and the graft,
forming with this thread a properly oriented loop,
releasing the thread from the second reserve, which has been drawn under the loops by the hooks, when the needle returns to the first part of the device.

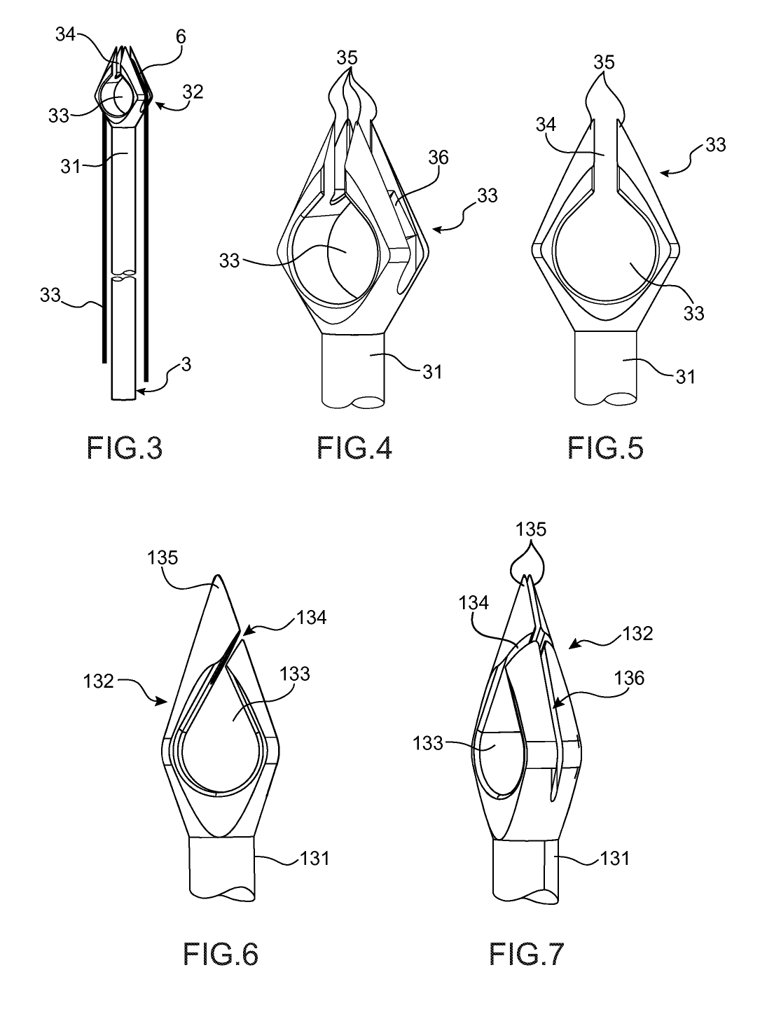

FIGS. 3 to 5 are different views of a needle usable by the device according to the invention. FIG. 3 is a three-quarter view of the needle equipped with the suturing thread of the first part. FIG. 4 is a three-quarter view showing the sharp end of the needle. FIG. 5 is a front view of the sharp end of the needle.

FIG. 3 shows the needle 3 equipped with the suturing thread 4. The needle 3 comprises a rod 31 having a non visible end intended to be fastened to the needle actuating means and having another end terminating in a sharp end 32 for piercing the walls of vessels. The sharp end of the needle includes a centre hole 33 communicating with the outside through a slot 34. The slot 34 is, as will be seen below, an outlet channel for the suturing thread of the second reserve. In this example, it is vertical, that is located in the axis of the needle.

FIGS. 4 and 5 better show the sharp end of the needle. It is observed that this sharp end includes four tips 35, all visible in FIG. 4. The tips 35 are defined on the one hand by the slot 34 and on the other hand by a kind of gutter 36, visible in FIG. 4, which intersects the slot 34 and which is intended to receive the loop 6 of the suturing thread 4 (see FIG. 3).

The gutter can be replaced by any other thread retaining form, enabling the thread to escape upon withdrawing the needles.

Other geometries for the sharp end of the needles are possible as shown in FIGS. 6 to 9.

FIGS. 6 and 7 are two different views of a same sharp end of a needle. These views show a needle rod 131 terminated by a sharp end 132 having a hole 133 communicating with the outside through a slot 134. In this example, the slot is located on the side with respect to the axis of the needle and enables, when the needle returns to the first part of the device, to let the thread go out of the second reserve. The sharp end comprises two tips 135 with the same size, separated by the gutter 136.

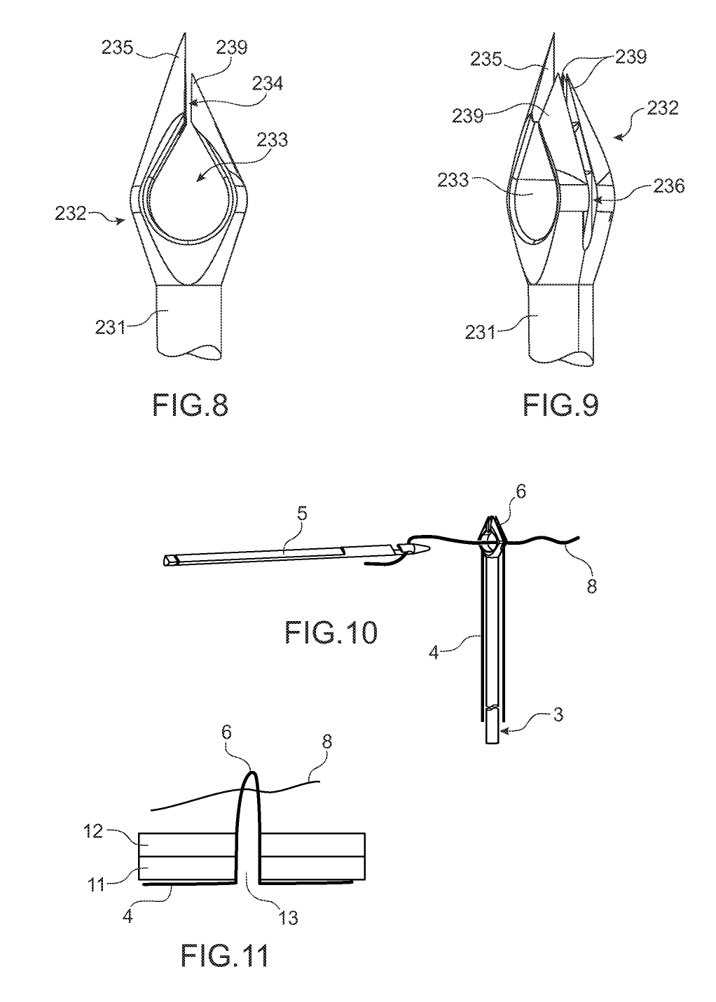

FIGS. 8 and 9 are different views of a same sharp end of a needle. These views show a needle rod 231 terminated by a sharp end 232 having a hole 233 communicating with the outside through a slot 234. In this example, the slot is vertical, that is located along the axis of the needle. The sharp end includes four tips defined on the one hand by the slot 234 and on the other hand by the gutter 236, visible in FIG. 9, which intersects the slot 234. In this example, the tips comprise a large tip 235, intended to initiate piercing of the walls, and three small tips 239.

FIG. 10 is an explanatory view representing the thread of the second reserve (or second thread) passing through a loop of the thread of the first reserve (or first thread). A needle 3 is represented as shown in FIGS. 3, 4 and 5, and supporting a loop 6 of the first thread 4. FIG. 10 illustrates the hook 5 returning to its initial position by drawing an end of the second thread 8, which corresponds to FIG. 2D.

FIG. 11 is another explanatory view showing in cross-section a part of the artery wall 11 placed to the side of a part of the graft wall 12. The reference 13 designates a hole provided in these walls by a needle of the device according to the invention. Withdrawing the needle has left in the graft a second thread 8 which has passed through the slot 34 (see FIG. 3). The second thread 8 retains the loop 6 of the thread 4 at the hole 13.

The device according to the invention enables an entire suture to be made with thread in a single manipulation cycle. Using threads enables tissues to be well healed. The invention also enables use of resorbable threads which disappear once the tissue is healed, thus leaving no foreign body within the patient.

The device according to the invention enables to pierce the vessel wall where the first part of the device is located (the first part is advantageously provided in an artery) from inside to outside. This is important in order to prevent atheromatous plaques of the inner wall of the diseased artery from detaching. The diseased artery can indeed have stenoses (plugs hampering the blood stream) and atheromatous plaques due to cholesterol build-up on the artery wall.

The device according to the invention enables an incision to be made between an artery and a graft to restore blood flow after the suture has been made, which enables to operate with beating heart without losing too much blood. Moreover, this enables section parts to be and remain well positioned with respect to one another, which promotes healing.

Both parts of the device can either be fastened to bodies of rigid instruments, or fastened to ends of instruments the ends of which are orientable (articulated or flexible), or fastened to ends of catheters so as to be able to access to the anastomosis site through natural routes (transluminal routes), for example through the femoral artery. The device thus enables to access areas difficult to reach, such as the back face of the heart, thanks to the low hindrance thereof

The principal application of the device according to the invention is that of making side-to-side anastomoses in mini-invasive surgery for aorto-coronary bypass operations. This application can be extended to mini-invasively suturing two hollow vessels or organs in side contact with each other.

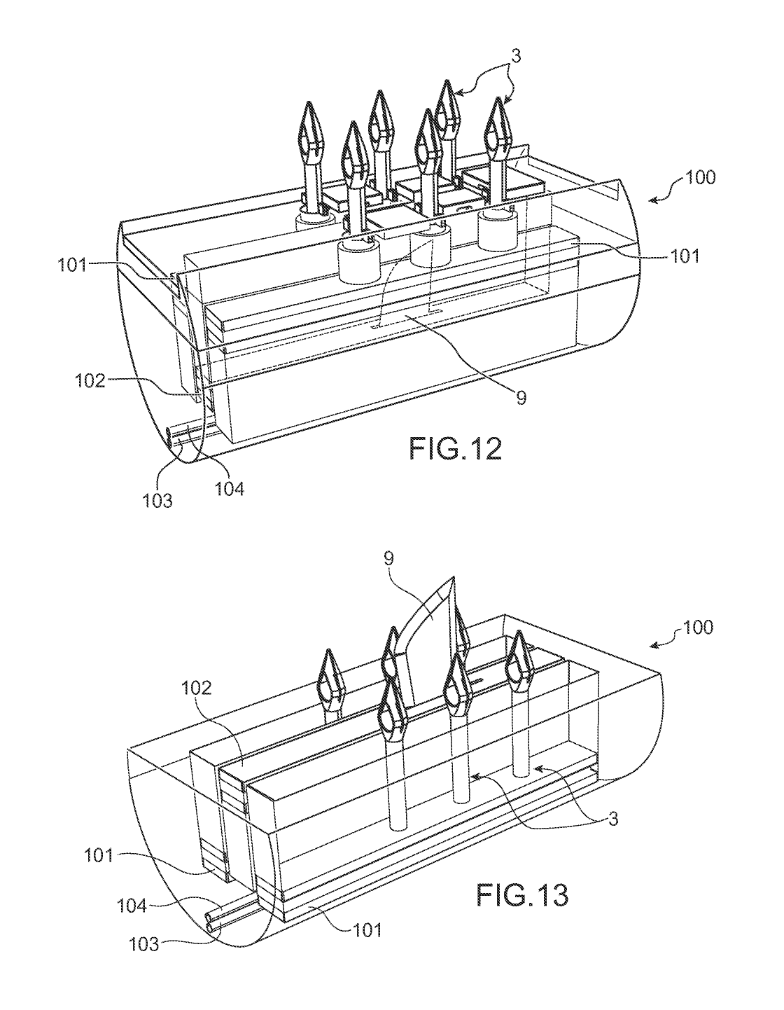

The first part and the second part of the device will now be shown while operating. This operation is illustrated by FIGS. 12 to 15 where the first and second parts are represented either separated, or together. The blood vessels (artery and graft) are not represented for simplification purposes.

FIG. 12 is a perspective view of a first part of the device according to the invention, showing the needles in the high position and the cutter in the low position. In this figure, the exterior shape of the first part 100 is seen. The first part comprises two rows of needles 3, each row of needles being driven by a hydraulic actuating mechanism 101. Between the hydraulic actuating mechanism of the needles, is provided a hydraulic actuating mechanism 102 of the cutter 9. This figure shows the needles 3 in the high position and the cutter 9 in the low position. Actuating the mechanisms 101 and 102 is caused by a fluid fed to the first part 100 via conduits 103 and 104. In this figure, the first suturing thread is not represented.

FIG. 13 is a perspective view of a first part of the device according to the invention, showing the needle 3 in the low position and the cutter 9 in the high position.

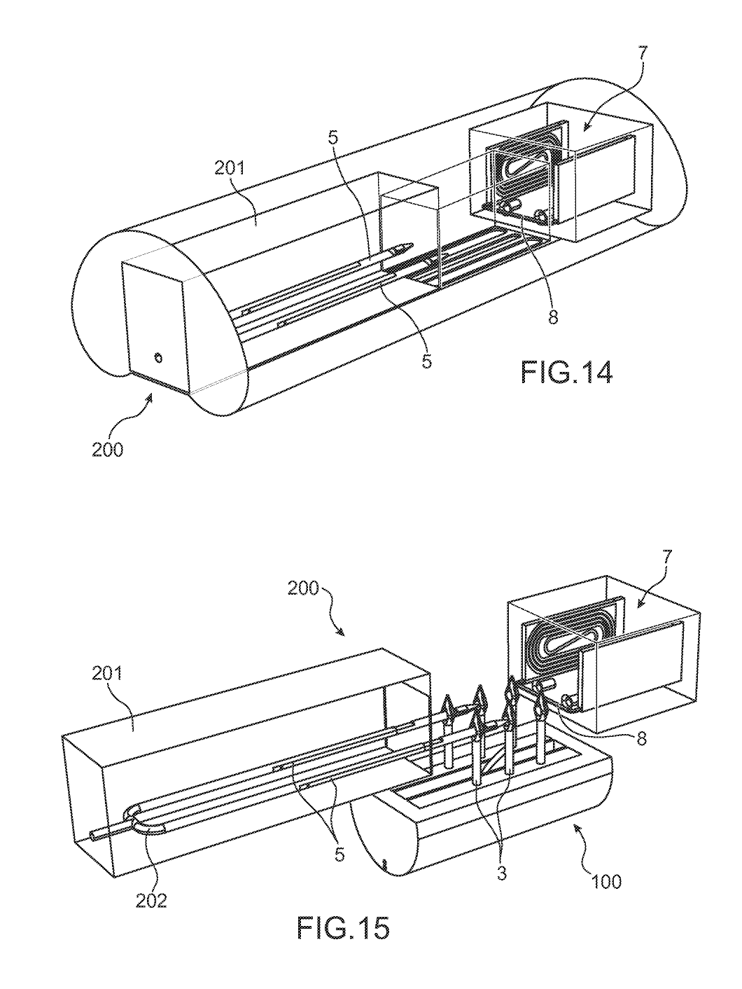

FIG. 14 is a perspective view of a second part of the device according to the invention, showing the second part during an operation phase. In this figure, the exterior shape of the second part 200 is seen. The second part 200 comprises a reserve 7 for the second suturing thread 8, two hooks 5 for passing through the holes of the sharp ends of two rows of needles of the first part, and a housing 201 of the hydraulic actuating mechanism of the hooks 5. FIG. 14 shows the hooks 5 before they catch the second thread 8.

FIG. 15 is a perspective view of some elements of the device according to the invention, illustrating the device during an operation phase. FIG. 15 shows the needles 3 in the high position (the non-represented walls of the vessels are thus pierced). The hooks 5, actuated by the hydraulic actuating mechanism 202 are seen when passing through the holes of the sharp ends of the needles to go and pick up the second suturing thread 8.

The diameters of each of both parts of the device according to the invention may have different sizes (without necessarily changing the other elements of each part) to be able to adapt to veins with different diameters and to different patients.

By varying the number of needles per row and the cutter size, different lengths of devices may be provided, which enables to make anastomoses with a different final perimeter (matching patients and veins with different diameters, requiring more or less blood flow rate).

Either or both parts of the device can be brought to the place of anastomosis by using natural routes (for example using a catheter) and/or be mounted to the end of manual and/or robotised laparoscopic instruments, as those from the Da Vinci robot, for various suturing applications, whether mini-invasive or not.

It is possible to replace flexible suturing threads by rigid, flexible or distortable threads (for example of shape memory alloy, for example of NiTi), with a complementary shape close to that of the suture.

A conventional laparoscopic hook may be used to pick ends of threads out of veins (by unfastening a suturing stitch on each row). Also, a single knot may be made with the four ends of the threads (or a clip may be used) to complete the anastomosis.

1. A device for suturing two hollow biological tissues bearing against each other, characterised in that it comprises a first part able to be provided on the free side of one of the two hollow biological tissues and a second part able to be provided on the free side of the other of the two hollow biological tissues, the first part and the second part cooperating together and comprising means for positioning them with respect to each other by grasping walls of the two hollow biological tissues bearing against each other,
the first part comprising:
a first reserve for a first suturing element,
needles aligned along two rows located on either side of an axis of the first part, the needles having their sharp ends directed to the second part, the sharp ends of the needles each having means for receiving a loop the first suturing element, each needle having a hole located so as to lie in the loop of the first suturing element received by the needle and substantially in the loop plane, the hole communicating with an exterior edge of the needle through a slot called outlet channel,
means for actuating the needles to bring them from a low initial position to a high final position, to pierce the walls of the two biological tissues through the sharp ends of the needles, and then to bring the needles back to their initial position,
the second part comprising:
a second reserve for a second suturing element,
driving means for driving a first part of the second suturing element into the holes of the needles in one of the two rows of needles and for driving a second part of the second suturing element into the holes of the needles of the other row of needles when the needles are in the high position.

2. The device according to claim 1, wherein the first part also comprises means for cutting the walls of the two biological tissues.

3. The device according to claim 2, wherein the cutting means are selected from an ultrasound device and a laser device.

4. The device according to claim 2, wherein the cutting means comprise a cutter the cutting edge of which is directed to the second part, the cutting means being located between the two rows of needles, the first part comprising means for actuating the cutting means to bring them from a low initial position to a high final position enabling the walls of the two hollow biological tissues to be incised by the sharp edge of the cutting means, and then to bring the cutting means back to the initial position.

5. The device according to claim 2, wherein the hollow biological tissues are vessels, the first part of the device has dimensions which make it able to be inserted in one of the vessels, the second part of the device has dimensions which make it able to be inserted in the other vessel, the device thus enabling a side-to-side anastomosis to be made between both vessels.

6. The device according to claim 1, wherein the means for positioning the first part and the second part with respect to each other comprise a female-shaped element formed in one of the parts and a male-shaped element formed in the other part.

7. The device according to claim 1, wherein the means for positioning the first part and the second part with respect to each other comprise a magnetic sensor accommodated in the first part and a magnetic sensor accommodated in the second part.

8. The device according to claim 1, wherein the means for actuating the needles comprise hydraulic actuating means.

9. The device according to claim 4, wherein the means for actuating the cutter comprise hydraulic actuating means.

10. The device according to claim 1, wherein the driving means for driving a first part and a second part of the second suturing element are actuated by hydraulic actuating means.

11. The device according to claim 1, wherein the driving means for driving a first part and a second part of the second suturing element comprise hooks able to draw the second suturing element.

12. The device according to claim 1, wherein the driving means for driving a first part and a second part of the second suturing element comprise pushers able to push the second suturing element.

13. The device according to claim 1, wherein the first suturing element is selected from a flexible thread, an elastic thread and a rigid thread.

14. The device according to claim 1, wherein the second suturing element is selected from a flexible thread, an elastic thread and a rigid thread.

15. A needle for use in the device according to claim 1, comprising a rod the first end of which is able to be fastened in the first part and the second end of which is sharp, the sharp end being provided with a hole, a gutter able to receive the first suturing element and made on the outline of the sharp end and substantially in the hole plane, and a slot communicating the hole with an exterior edge of the needle, the sharp end comprising at least two tips.

16. The needle according to claim 15, wherein the slot is made in the axis of the rod, whereby the sharp end comprises four tips.

17. The needle according to claim 15, wherein the slot is not made in the axis of the rod, whereby the sharp end comprises two tips.

18. The needle according to claim 15, wherein at least one of the tips is longer than the others.
